(19) United States
(12) Patent Application Publication
(10) Pub. No.: US 2021/0214660 A1
(43) Pub. Date: Jul. 15, 2021

(54) A System and Method for Growing and Extracting Algae

(71) Applicant: Shalem for Space Industries Ltd., Kfar Ruppin (IL)

(72) Inventor: Jacob GURMAN, Kfar Ruppin (IL)

(73) Assignee: Shalem for Space Industries Ltd., Kfar Ruppin (IL)

(21) Appl. No.: 17/250,125

(22) PCT Filed: Jun. 6, 2019
(86) PCT No.: PCT/IL2019/050645

§ 371 Date: Dec. 1, 2020

(30) Foreign Application Priority Data
Jun. 7, 2018 (IL) 259890

(51) Int. Cl. C12M 1/00; C12M 1/36; C12M 1/26; C12M 1/34; C12N 1/12

(52) U.S. Cl. CPC C12M 21/02; C12M 41/48; C12M 33/00; C12M 29/00; C12M 29/22; C12M 41/12; C12N 1/12; C12M 41/34; C12M 41/36; C12M 41/44; C12M 29/04; C12M 39/00; C12M 41/26

(57) ABSTRACT

A system adapted for growing and harvesting algae and for obtaining products thereof adapted for domestic use, comprises a Photo-bioreactor adapted for use in a home environment, said Photo-bioreactor being controlled by a CPU control unit, said Photo-bioreactor being coupled to a Separation Harvesting and Extraction Unit (SEU), adapted to receive algae from said Photo-bioreactor as an input, and to generate therefrom an algal paste, said SEU being in turn coupled to an extraction unit adapted to receive said paste and to extract a desired material therefrom, said system being controlled and monitored by a CPU control unit.

### FIELD OF INVENTION

The present invention relates to the field of single cell microalgae/bacteria photo bioreactors. More specifically the invention relates to a system for growing microalgae/bacteria, harvesting and extracting materials from said microalgae/bacteria.

### BACKGROUND OF INVENTION

Photo bioreactors are vessels which incorporate some type of light source and carry out a biological reaction and are used to culture aerobic cells for conducting cellular or enzymatic immobilization.

However, the dimensions of bioreactors are usually so big that they may even be larger than a typical house room. In addition, the harvesting of the algae and extracting materials (e.g., different chemical compounds) from the algae are processes that are done in industrial labs and usually take time and human control, which makes the procedure long and cumbersome. This makes it almost impossible for people who wish to grow algae at home to do so in practice. Therefore, there is a need in the field to provide a system, sized and customized for home/personal/family use, that incorporates different functions: the growing, harvesting and the extraction of materials in one system.

It is an object of the present invention to provide a system for both growing and extracting materials from the algae in one system.

Other objects and advantages of the invention will become apparent as the description proceeds.

### SUMMARY OF THE INVENTION

The invention is directed to a system adapted for growing and harvesting algae and for obtaining products thereof adapted for domestic use, comprising a Photo-bioreactor adapted for use in a home environment, said Photo-bioreactor being controlled by a CPU control unit, said Photo-bioreactor being coupled to a Separation Harvesting and Extraction Unit (SEU), adapted to receive algae from said Photo-bioreactor as an input, and to generate therefrom an algal paste, said SEU being in turn coupled to an extraction unit adapted to receive said paste and to extract a desired material therefrom, said system being controlled and monitored by a CPU control unit.

According to one embodiment there is provided a system for automatically growing and harvesting algae and extracting materials from said algae, for domestic use, comprising:
a. a small dimension Photo-bioreactor with a dedicated water container for supplying optimal environment for algae growth, said Photo-bioreactor being controlled by a CPU control unit;
b. a Separation harvesting and Extraction Unit (SEU), which receives algae from said Photo-bioreactor as an input, crushes the algae and filters said crushed algae to receive a fresh paste of algae, wherein said fresh paste is outputted to a user upon request, or outputted for a specific extraction of materials where said fresh paste is extracted by an extraction unit to receive said materials and output said extracted materials to said user; and
c. a CPU control unit which controls and monitors said Photo-bioreactor and said SEU unit and is provided with a user interface to receive instructions from the user; wherein said CPU control unit receives data from said Photo-bioreactor and said SEU unit, analyses the data and provides commands to said Photo-bioreactor and said SEU unit to keep the photo-bioreactor balanced with optimal environment for algae growth and to monitor the extraction of algae in the SEU according to the user instructions.

In an embodiment, the Photo-bioreactor comprises:
a. a sensors network for sensing the environment in said water container;
b. an air pump which inserts CO₂ through an air opening to the dedicated water container and extracts oxygen from the water container to the outside, and an air valve for opening and closing said air opening;
c. a light source for providing light conditions to the algae;
d. a heating unit;
e. a nutrient supply means adapted to releases nutrients in a controlled manner according to the algae needs, and which can be filled and replaced as reusable means;
f. water insertion and compensation mean, adapted to insert water to the photo-bioreactor according to the needs of the algae.

In another embodiment, the Photo-bioreactor further comprises a secondary water container, for secondary growth of algae and manipulation or stress to the algae to increase values of nutrition.

In an exemplary embodiment, the physical dimensions of the small dimension photo-bioreactor are between 35 cm×35 cm×45 to 45 cm×45 cm×55 cm.

In an embodiment, the sensors in the Photo-bioreactor are from the list of: turbidity meter, pH sensor, CO₂ sensor, OXD sensor, conductivity sensor, water/volume sensor and temperature sensor.

In an embodiment, the Separation and Extraction Unit comprises:
a. a filtering unit for disrupting the algae received from the Photo-bioreactor and filtering medium from the disrupted algae and outputs a fresh paste media;
b. an extraction unit for extracting specific materials from the algae; said extraction unit receives the fresh paste, extracts the paste and output the requested materials;
c. a cleaning and rinsing unit for cleaning the separation and extraction unit after the extraction process of the algae is over.

In an embodiment, the separation and extraction unit further comprise a drying unit for drying and dehydrating algae.

In another embodiment, the extraction unit comprises vibrating balls for extracting the paste of algae.

In a further embodiment, the extraction unit comprises a freezing and thawing unit for extracting the paste of algae.

In still another embodiment, the extraction unit comprises an ultra-sonic wave unit for extracting the paste of algae.

In yet another embodiment, the extraction unit comprises a centrifuge for extracting the paste of algae.

In still a further embodiment, the CPU control unit is connected to an external data base which receives data from all the systems in use, and wherein the CPU control unit can be remotely controlled and updated by an administrator.

### BRIEF DESCRIPTION OF THE DRAWINGS

FIG. 1 is a schematic block diagram of the system according to an embodiment of the invention;

FIG. 2 schematically shows a block diagram of the system of the bioreactor according to an embodiment of the invention;

FIG. 3 schematically shows a block diagram of the Separation and Extraction Unit of the system according to an embodiment of the invention;

FIG. 4 schematically shows a block diagram of the CPU Control Unit of the system according to an embodiment of the invention;

FIG. 5 is a block diagram of the fluidic system according to one embodiment of the invention;

FIG. 6 is a block diagram of the control system according to one embodiment of the invention;

FIG. 7 (a, b) is a transparent view of a system of the invention, showing some main elements thereof; and

FIG. 8 is a detail of an algal filter according to the embodiment of FIG. 7.

### DETAILED DESCRIPTION OF EMBODIMENTS OF THE INVENTION

The device presented herein is adapted to grow various types of microalgae, certain algae, cyanobacteria, and bacteria. For the sake of brevity, the term algae used herein refers to various types of microalgae, certain algae, cyanobacteria, and bacteria.

The system described below is a device for domestic use with relatively small dimensions. An illustrative and non-limitative example of suitable dimensions for a device of the invention is approximately between 35 cm×35 cm×40 cm to 45 cm×45 cm×55 cm, so as to be easily stored at a domestic kitchen like a water bar. As will be easily apparent to the skilled person, such dimensions only serve to illustrate the invention and apparatus having different dimensions can be devised without exceeding the scope of the invention.

In an embodiment, the system may be adapted to be dedicated to grow and extract one type of algae. In another embodiment, the system may be adapted to grow and extract more than one type of algae.

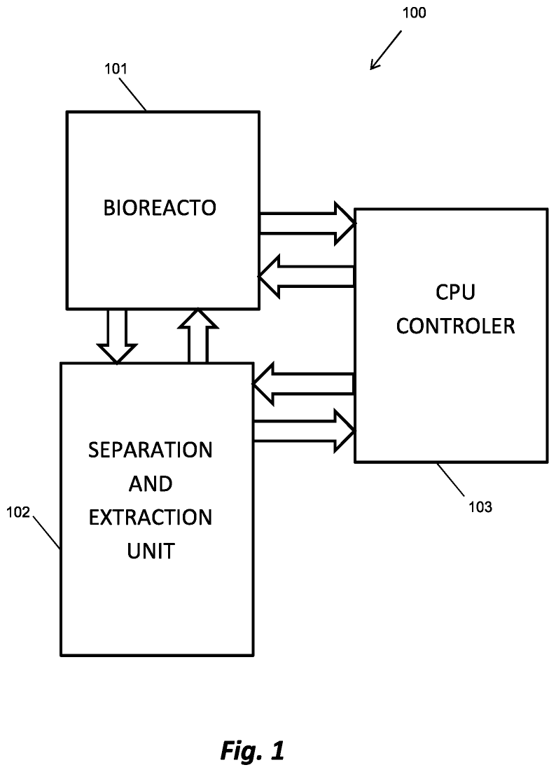

FIG. 1 schematically describes the system according to an embodiment of the invention. System 100 comprises three main units: A bioreactor 101, which is a photo-bioreactor for growing various types of microalgae, certain algae, cyanobacteria, and bacteria. This unit is connected to a Separation, Harvesting and Extraction Unit (SEU) 102, and both units are connected to a CPU control unit 103, which monitors, controls and optimizes the system's activity. The CPU control and communication unit comprises a user interface for enabling a user to interact with the system, request for specific extraction or to access data and intervening the control process. The user interface may be a touch screen or any other suitable mean for interfacing with the system.

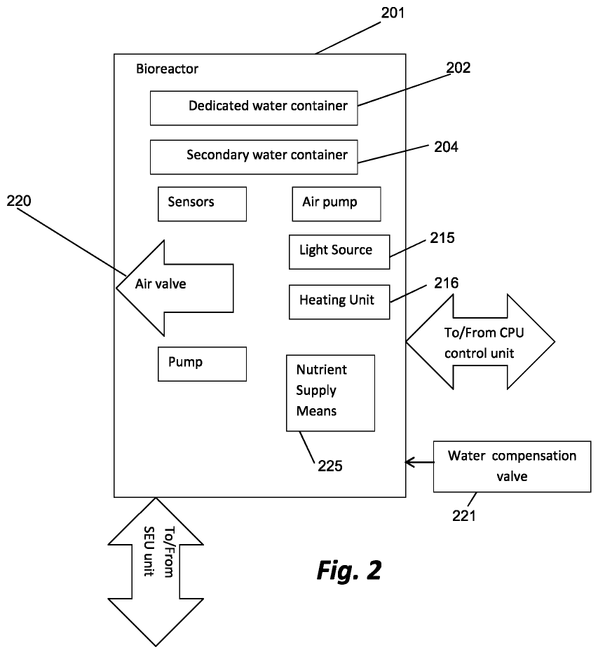

FIG. 2 schematically describes the Photo-bioreactor 201 of the system according to an embodiment of the invention. Bioreactor 201 comprises a dedicated water container 202, for growing the algae. The container keeps the system balanced by maintaining all the algae needs for optimal growth. The lid of the container 202, and the lid's edges have openings designed to connect the peripheral equipment that supports the growth.

In an embodiment, a secondary water container, 204, allows secondary growth and manipulation of the algae, which causes stress to algae by radically changing the growth environment, (for example: causing stress conditions such as nitrogen starvation and/or high salinity and/or light change, could stimulate synthesis and accumulation of bio compounds such as lipids, protein or sugars). In this way the system enables certain types of algae to increase values of nutrition which are of interest, and/or increase the percentage of certain components of algae before the refining and/or extraction.

Bioreactor 201 contains several sensors for collecting data in real-time about the algae and its growth condition, and the data collected is transferred to the CPU controller unit. Each microalga has a different ideal growth condition such as: temperature, pH level, conductivity etc. The control unit monitors the growth of each type of algae and optimizes the growth condition, according to the type of algae and the data collected from the sensors in the bioreactor. Once the data is processed and analyzed, the controller unit transmits commands to the bioreactor of the system to adjust the condition of growth, for example: to reduce water bubbles, to change oxygen and/or CO₂ (Carbon dioxide) levels, to change lighting, temperature, adding nutrients, etc. The sensors in the bioreactor may be a OXD (dissolved oxygen), a CO₂ sensor, which is used to monitor the CO₂ levels in the photo-bioreactors in real time, since the algae consume CO₂ in a photo-synthesis process, a pH sensor, which monitors the level of PH, a conductivity sensor, a temperature sensor, a turbidity meter and a volume/water level sensor for measuring the volume of the water in the dedicated water container. The temperature sensor measures the temperature in the Photo-bioreactor and transmits the results to the CPU control unit which compares the real time temperature to the optimal temperature required for the algae. If the temperature is lower than the optimal temperature, the controller turns on the heating unit 216. The volume/water level sensor informs the CPU controller unit of water loss, either because of evaporation or because of filtering algae in favor of production in the filtration and extraction unit. The CPU controller unit compensates for the loss of water by opening a water compensation valve 221. The water compensation valve 221, opens or closes to return the necessary level of water. The water level sensor informs the system when the amount of water is optimal, to stop the water flow. Bioreactor 201 also comprises an air pump and an air valve, which are controlled by the CPU controller unit, and which enable air flow, or optionally filtered air flow for mixing of water, algae, and nutrients, as well as oxygen regulation, CO₂ regulation, temperature and more. The bioreactor comprises a light source 215 (usually a Led) which provides illumination for optimum photosynthesis of the algae, and which is controlled by the CPU control unit.

A heating unit 216 is located at the bioreactor and is activated by the CPU control unit, according to the temperature sensor, to maintain optimal temperature, for the growth of the algae. Air valve 220 in the bioreactor enables the release of vapor.

A drainage port material and valve, improves material exits for filtering or directly for external drainage, if such is required. In this example, the CPU control unit determines the amount of the container emptying and will distribute accordingly in water and nutrients.

A nutrient supply means 225 is adapted to selectively release nutrients to the water container in a controlled manner according to the algae needs, and which can be filled and replaced as a reusable unit, or provided as a disposable unit. The nutrient supply is controlled by the CPU controller.

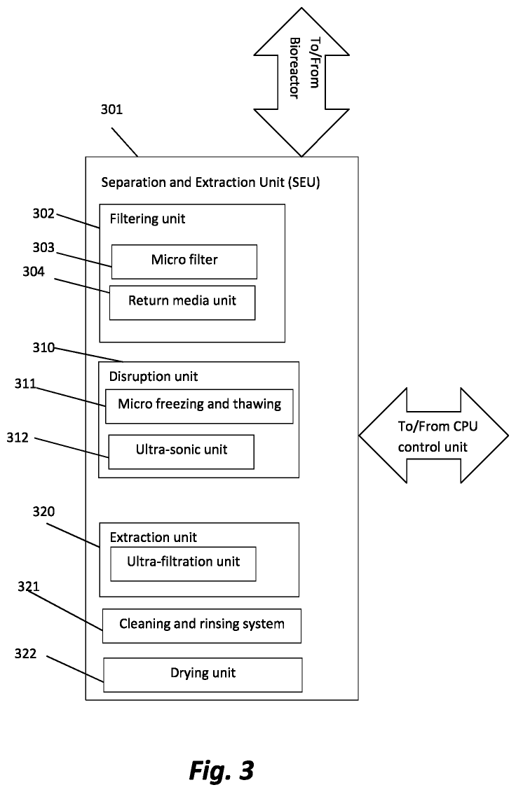

FIG. 3 schematically shows the Separation and Extraction Unit (SEU) 301 of the system according to one embodiment. Part of the SEU's, runs on demand. By using the touch screen of the system, the user can ask for a daily dose of algae, the SEU unit will provide this daily dose of algae in the form of fresh paste, as well as in dried form. Moreover, the SEU 301 can give, according to the algae type, a dose of unique extracted compound, by choosing from the system's display to enjoy a specific extraction, for example extraction of protein or polysaccharides. The special extraction is done by the disruption units 310, and the filtering unit 302. In the SEU there is a filtering unit 302, which comprises a micro filter 303, which is a micronized mesh whose size is determined by the type of algae (typically between 5-40 micron), and a paste output valve, which provides fresh algae paste output if requested by the user, or passes the fresh algae paste to the disruption unit 310 or to the Extraction Unit 320, for further special extraction. In the case that the user wants to consume the algae paste, the system can extract the algae at high concentration depending on the type of algae (whether it is Chlorella or Donalella, Haematococcus pluvialis, Spirulina or other, at the level of thick liquid to paste). The Filtering Unit also comprises a Returns Medium Unit 304, which returns the water with the nutrients that were used during the growth and extraction process to the Photo-bioreactor valve, for reuse of the enhanced water.

The SEU comprises a disruption unit 310, with a micro freezing and thawing unit 311, for disruption of the algae at the special extraction process. The disruption is done optionally by small balls or by cooling the algae to low temperature and then thawing the algae or by ultra-sonic wave unit. The term “disruption”, as used herein, refers to various processes involving processing and/or breaking of the algal matter into smaller parts.

In an embodiment, there is also an ultra-filtration system with a centrifuge for special extraction of materials from the algae.

A cleaning and rinsing system 321, which is an automatic self-cleaning system based on water connection, is activated after the extraction process is over, to clean the SEU from all residual algae and to clean the SEU for the next extraction process. The residual algae may be stored for future use.

An optional unit is a drying unit 322, which receives the biomass after filtration, dehydrates the algae, and stores the dried material in a designated container for future use. The dried algae can be stored for a long term. The CPU control unit detects the status of algae growth when it exceeds the capacity of the Photo-bioreactor and in the absence of precipitation/consumption of algae from the bioreactor, the CPU control unit automatically sends a command to the bioreactor to pass material for filtering and drying to the drying unit, thereby achieving both objectives of controlling and regulating the growth of the algae and keeping excess algae in its dry form so that it can be used later and can be stored for a long term.

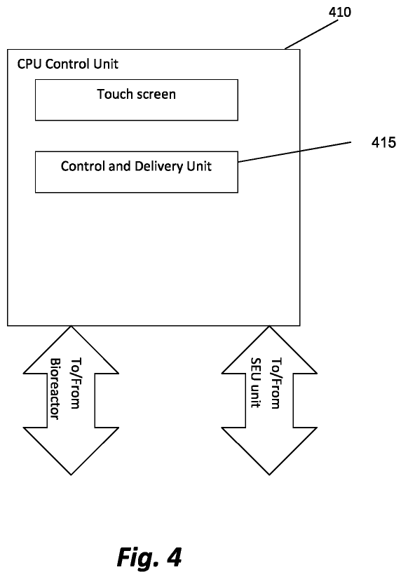

FIG. 4 schematically shows the CPU control unit 410, of the system according to an embodiment. The CPU control unit monitors the bioreactor 201, and receives and communicates all the growth data collected from the sensors within the bioreactor, for example: the water level/volume, temperature, pH level, conductivity, etc. The control unit analyzes the data received from the sensors in real time and makes decisions based on the received data, whether to intervene in the growth of the algae or not. For example: whether to raise the temperature add salt or any other nutrient to balance the system to be in optimal condition for the growth of the algae.

All this information is recorded and can be communicated to a central analysis center.

The CPU control unit also controls and monitors the SEU unit and the delivery of material between the Photo-bioreactor and the SEU unit for extraction of materials from the algae and delivery of medium from the SEU unit to the bioreactor for reuse, such as enhanced water that were left at the end of a harvest process.

The control unit comprises a user-friendly interface touch screen 415. Through the touch screen the user can get access to the data saved in the CPU control unit, and also to manually insert data if necessary or even to manually intervene and send commands to the end units of the Photo-bioreactor and/or the SEU unit. The user may get daily dose of protein extraction, polysaccharides as fresh paste or dry matter. The control unit is connected to an external data base which receives online or batch logs. The CPU control unit can be remotely controlled and updated by a remote administrator.

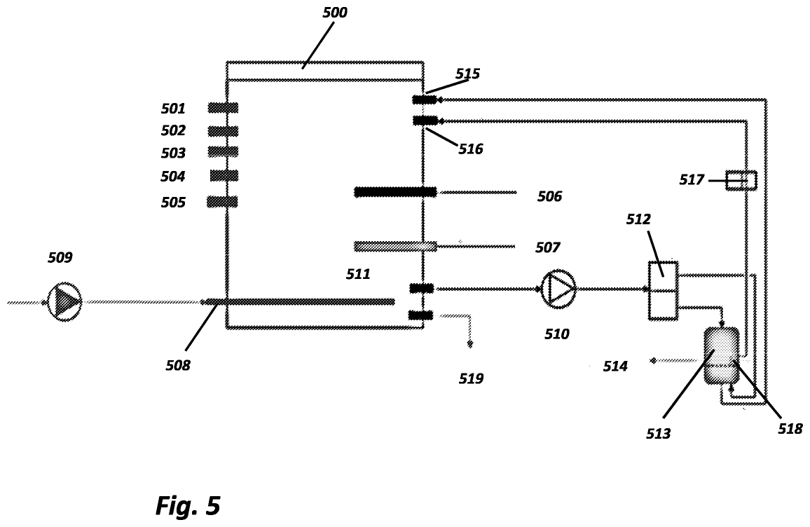

Looking now at FIG. 5, a block diagram of the fluidic system according to one embodiment of the invention is shown. Water tank 500 can be made of a transparent material e.g. polycarbonate or acrylic, and is provided with the following sensors: level sensor 501, O₂ sensor 402, pH sensor 503, conductivity sensor 504 and temperature sensor 505. Of course, the number and types of sensors can vary in different embodiments and the above list of sensors is provided only for the purpose of illustration.

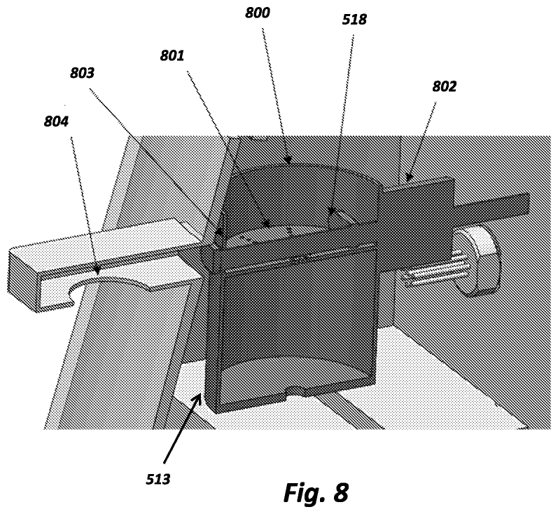

The water tank in which the algal growth takes place is further provided with one or more heater elements 506 and lighting elements 507, as well as bubble tube 508 through which air and thus oxygen are supplied to the tank through air pump 509. A water pump 510 draws the liquid containing algal material from outlet 511 and, in this particular embodiment, feeds it to a three-way valve 512 which feeds a filter 513 through which the material passes before being forwarded to a dispense port 514 (not shown). Material that does not pass the filter is recycled to the tank 500 via return port 515, and additional overflow material can be returned to tank 500 via return port 516, if the on/off valve 517 is open. A wiper 518 is provided for wiping solids that accumulate on the filter mesh, as further described with reference to FIG. 8. Water can be released from the tank via drain 519.

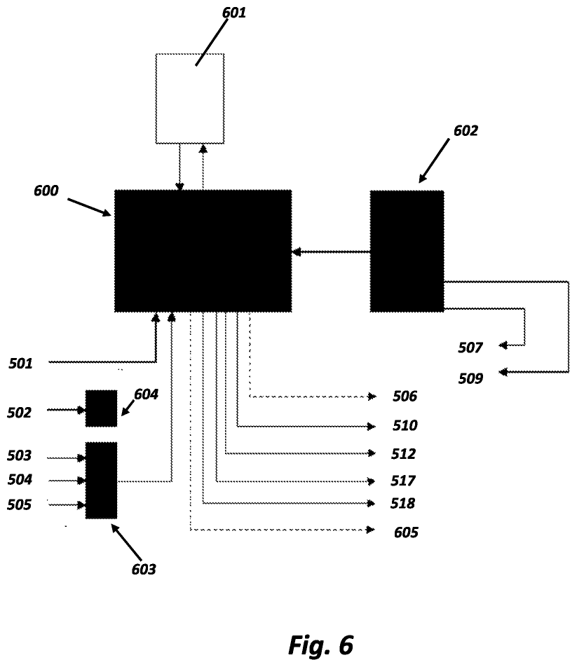

FIG. 6 illustrates the operation of the system controller according to one embodiment of the invention. Main controller 600 can be operated, for instance, via a touch screen display 601, and receives power from power supply 602, which may also supply power to lighting elements 507 and to air pump 509. Controller 600 receives data input from sensors 501, and from sensors 503-505 through transducer 603, while oxygen sensor 502 is independently controlled by controller 604. Controller 600 activates and operates the following elements: heating elements 506, water pump 510, valve 512, on/off valve 517, wiper motor 518, and nutrient system supply 605 (not shown).

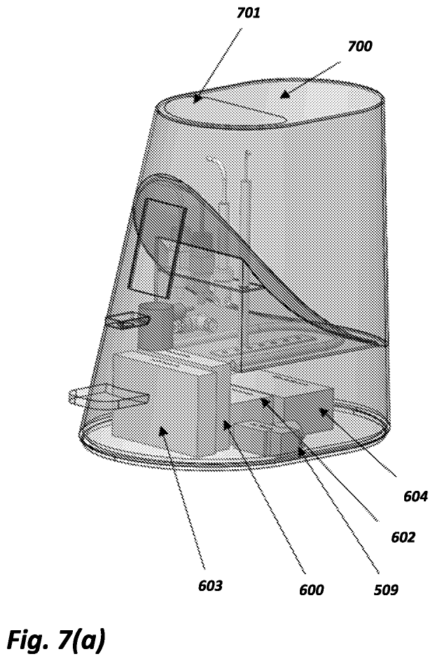

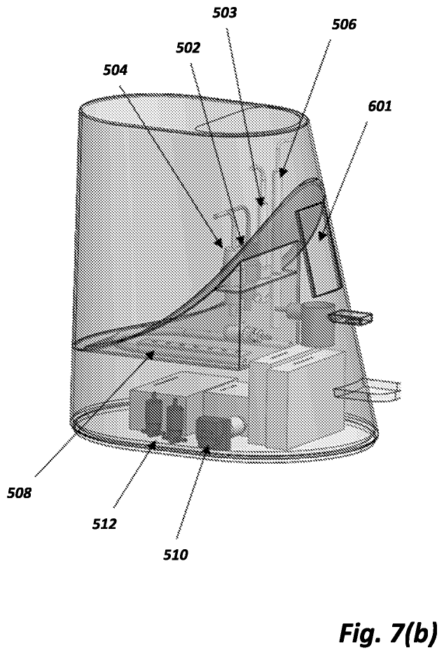

FIG. 7 (a, b) are two transparent perspective views of a system according to one embodiment of the invention, showing some main elements thereof. The various elements of the system are numbered as in FIGS. 5 and 6, and their description is therefore not repeated here, for the sake of brevity.

FIG. 8 is a detail of filter 513 of FIG. 5. Filter 513 consists of filter housing 800, a filter mesh 801, a wiper motor 802 that operates wiper 518, a seal 803, and outlet port 804.

Of course, in the above description only some main elements of the system have been shown for the purpose of illustrating the invention, but as will be apparent to the skilled person, additional and/or alternative elements may be provided in alternative embodiments of the invention. The above description has been provided for the purpose of illustration and is not intended to limit the invention in any way except as provided for in the appended claims.

1. A system adapted for growing and harvesting algae and for obtaining products thereof adapted for domestic use, comprising a Photo-bioreactor adapted for use in a home environment, said Photo-bioreactor being controlled by a CPU control unit, said Photo-bioreactor being coupled to a Separation Harvesting and Extraction Unit (SEU), adapted to receive algae from said Photo-bioreactor as an input, and to generate therefrom an algal paste, said SEU being in turn coupled to an extraction unit adapted to receive said paste and to extract a desired material therefrom, said system being controlled and monitored by a CPU control unit.

2. A system according to claim 1 for automatically growing and harvesting algae and extracting materials from said algae, for domestic use, comprising:
a. a small dimension Photo-bioreactor with a dedicated water container for supplying optimal environment for algae growth, said Photo-bioreactor being controlled by a CPU control unit;
b. a Separation harvesting and Extraction Unit (SEU), which receives algae from said Photo-bioreactor as an input, crushes the algae and filters said crushed algae to receive a fresh paste of algae, wherein said fresh paste is outputted to a user upon request, or outputted for a specific extraction of materials where said fresh paste is extracted by an extraction unit to receive said materials and output said extracted materials to said user; and
c. a CPU control unit which controls and monitors said Photo-bioreactor and said SEU unit and is provided with a user interface to receive instructions from the user; wherein said CPU control unit receives data from said Photo-bioreactor and said SEU unit, analyses the data and provides commands to said Photo-bioreactor and said SEU unit to keep the photo-bioreactor balanced with optimal environment for algae growth and to monitor the extraction of algae in the SEU according to the user instructions.

3. The system of claim 2, wherein the Photo-bioreactor comprises:
a. a sensors network for sensing the environment in said water container;
b. an air pump which inserts CO₂ through an air opening to the dedicated water container and extracts oxygen from the water container to the outside, and an air valve for opening and closing said air opening;
c. a light source for providing light conditions to the algae;
d. a heating unit;
e. a nutrient supply means adapted to releases nutrients in a controlled manner according to the algae needs, and which can be filled and replaced as reusable means;
f. water insertion and compensation mean, adapted to insert water to the photo-bioreactor according to the needs of the algae.

4. The system of claim 3, wherein the Photo-bioreactor further comprises a secondary water container, for secondary growth of algae and manipulation or stress to the algae to increase values of nutrition.

5. The system of claim 2, wherein the small dimension photo-bioreactor is between 35 cm×35 cm×45 to 45 cm×45 cm×55 cm.

6. The system of claim 2 wherein the sensors in the Photo-bioreactor are from the list of: turbidity meter, pH sensor, CO₂ sensor, OXD sensor, conductivity sensor, water/volume sensor and temperature sensor.

7. The system of claim 2, wherein the Separation and Extraction Unit comprises:
a filtering unit for disrupting the algae received from the Photo-bioreactor and filtering medium from the disrupted algae and outputs a fresh paste media;
an extraction unit for extracting specific materials from the algae; said extraction unit receives the fresh paste, extracts the paste and output the requested materials;
a cleaning and rinsing unit for cleaning the separation and extraction unit after the extraction process of the algae is over.

8. The system of claim 7, wherein the separation and extraction unit further comprise a drying unit for drying and dehydrating algae.

9. The system of claim 7, wherein the extraction unit comprises vibrating balls for extracting the paste of algae.

10. The system of claim 7, wherein the extraction unit comprises a freezing and thawing unit for extracting the paste of algae.

11. The system of claim 7, wherein the extraction unit comprises an ultra-sonic wave unit for extracting the paste of algae.

12. The system of claim 7, wherein the extraction unit comprises a centrifuge for extracting the paste of algae.

13. The system of claim 1, wherein the CPU control unit is connected to an external data base which receives data from all the systems in use, and wherein the CPU control unit can be remotely controlled and updated by an administrator.
